(19) United States
(12) Patent Application Publication
(10) Pub. No.: US 2010/0292807 A1
(43) Pub. Date: Nov. 18, 2010

(54) Stabilising Knee Joint for a Lower Limb Prosthesis

(76) Inventors: Juan Jose Ochoa Velez, Medellin (CO); Mir Saeed Zahedi, London (GB)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/744,081

(22) PCT Filed: Nov. 14, 2008
(86) PCT No.: PCT/GB2008/003860

§ 371 Date: May 21, 2010

(30) Foreign Application Priority Data
Nov. 21, 2007 (GB) 0722837.2
Jul. 17, 2008 (GB) 0813132.8

(51) Int. Cl. A61F 2/68; A61F 2/64

(52) U.S. Cl. 623/44; 623/43

(57) ABSTRACT

In a stabilising knee joint for a lower limb prosthesis, weight-activated knee stabilisation is achieved by interen-gagement of a brake disc (32) associated with an upper knee chassis (10) and a brake member (34) associated with a shin carrier (18). The knee chassis is mounted on a posterior end portion (14P) of a resiliency biased activation arm (14) which pivots on the carrier (18). To provide a mechanical advantage in the brake action, a circumferential portion of the brake disc (32) is tapered, and the brake member (34) has a correspondingly tapered channel which receives the circumferential disc portion. The arrangement of the brake members (32, 34) and their mounting on the shin carrier (18) is such that substantial release of frictional engagement of the brake members can be substantially achieved even when a knee flexion moment is applied to the joint.

This invention relates to a stabilising knee joint for a lower limb prosthesis having a brake rotor on one of the components and a brake member on the other component, the brake rotor and the brake member having matching inclined surfaces that interengage frictionally in response to relative approaching movement of the upper and lower components associated with a compressive load being applied to the knee joint.

Such a knee joint is disclosed in WO97/10781 which teaches a brake rotor in the form of a tube extending in the medial-lateral direction and centred on the knee axis, the tube being externally screw-threaded with the flanks of the threads being inclined to a plane perpendicular to the knee axis. A resiliently deformable brake member that is internally screw-threaded surrounds the screw-threaded tube. When the knee joint is subject to a compressive load, the surrounding brake member is contracted so that the interengaging inclined surfaces of the screw threads engage frictionally to resist flexion of the joint. Users have found that a lower limb prosthesis incorporating such a knee joint can be difficult to control. The result is an unnatural gait pattern at the end of the stance phase.

It is an object of the invention to provide a stabilising knee joint that has improved functionality.

According to a first aspect of the invention, a stabilising prosthetic knee joint for a lower limb prosthesis, comprises an upper component for attachment to a thigh part and a lower component for attachment to a shin part, the upper component being pivotable relative to the lower component, wherein the joint further comprises a first brake member that moves with one of the components and a second brake member associated with the other component, the first and second brake members having opposing surfaces, and wherein the first brake member is mounted so as to be movable towards the second brake member against resilient biasing means in response to the application of a compressive force to the joint to cause the first brake member to bear against the second brake member whereby frictional inter-engagement of the opposing surfaces resists flexion of the joint, and wherein the mechanism of the joint is so configured that a resultant force imposed on the first brake member by the second brake member when the said compressive force is applied to the joint acts in a direction that allows substantial release of the said frictional engagement when the compressive force is removed, even when a flexion moment is applied to the joint.

According to a second aspect of this invention, a stabilising prosthetic knee joint for a lower limb prosthesis comprises an upper component for attachment to a thigh part and a lower component for attachment to a shin part, the upper component being pivotable relative to the lower component about a knee axis, wherein the joint further comprises a first brake member that moves with one of the components and a second brake member associated with the other component, the first and second brake members having opposing surfaces, and wherein the first brake member is pivotally supported and resiliently biased such that when a compressive force is applied to the joint, the first brake member bears against the second brake member whereby frictional inter-engagement of the opposing surfaces resists flexion of the joint, and such that subsequent removal of the compressive force whilst a flexion moment is applied to the joint causes substantial release of the said frictional engagement.

The mechanism of the joint is preferably configured so as to convert an activation force into an engagement force with a mechanical advantage the engagement force causing the first brake member to bear against the second brake member. In particular, the mechanical advantage may be produced as a result of the configuration of the brake members and/or the manner in which they are supported. The mechanical advantage is advantageously produced substantially independently of any flexion moment applied to the joint and such that the activation force is the dominant component, at all times during use of the joint, of the total force causing the brake members to bear against each other. In many prior devices, including the joint disclosed in WO97/10781, the flexion moment resulting from application of a compressive load on the joint when flexed or partly flexed is used to increase the frictional engagement force between the brake members. Unless such assistance is very carefully controlled, it can result in locking of the joint against flexion when the compressive load is reduced or, in some cases, when the compressive load is removed altogether at the end of the stance phase. This is contrary to the need to start flexion of the joint at the end of the stance phase under the effect of both a flexion moment and a compressive force.

In the preferred embodiment of the present invention, the mechanical advantage is produced at least in part by the inclined orientation of the opposing surfaces of the brake members. In this preferred embodiment, one of the brake members is a brake disc that is of tapered cross-section and the other brake member is a channel member or shoe having a channel defined between jaws which have disc-contacting surfaces substantially parallel to the respective side surfaces of the disc. The knee joint is preferably monocentric, having a fixed knee axis of rotation defined by the axis of rotation of the upper component with respect to the lower component. The brake disc is conveniently centred on the knee axis of rotation. A compact arrangement can be achieved if the brake disc rotates with the upper component of the joint.

Resilient support of the first brake member may be achieved by mounting it on a resiliently deflectable activation arm, the orientation and mounting of the arm, as well as that of the brake members, being such that when a compressive force is applied to the joint, the arm is deflected so as to bring the opposing surfaces of the brake members into frictional engagement in order to resist flexion of the joint.

In addition, the second brake member, i.e., preferably the channel member or brake shoe, is mounted in one of the upper and lower components. The second brake member may be rigidly mounted in the respective component, or it may be resiliently mounted, being biased against a stop in the absence of the said compressive load. If resiliently mounted, the second brake member may be pivotally attached to the said component, the pivotal mounting defining a brake member pivot axis that is substantially parallel to the knee axis and is spaced from a first line passing through the knee axis and the centroid of the disc-contacting surfaces.

The activation arm may be pivotally mounted on the same component as the channel member and has an outer end portion that carries the disc and the other one of the upper and lower components. In this case, the included angle (hereinafter referred to as the “activation angle”) between (a) a second line passing through the axis of rotation of the disc on the activation arm (the knee axis) and the pivot axis (the activation axis) of the activation arm and (b) the first line is between 40° and 90° when the second brake member engages the disc. In the preferred embodiment, the activation arm is pivotally connected to the lower component of the joint about the activation axis. The upper component is pivotally connected to the other end of the activation arm about the knee axis. The activation and the knee axes define the corresponding activation and knee centres at their intersection with the mid plane of the joint. A first segment is defined on the mid-plane of the joint between said two centres.

In the preferred embodiment the second brake member is pivotally mounted on the activation arm and is resiliently supported on the lower component. This second member defines a second segment, between the knee centre and the projection onto the mid-plane of the centroid of the area of contact between first and second brake members. In this embodiment, it is the configuration of axes and brake members that are such as to define a geometry in which the angle between said first and second segments (the “activation angle”) is between 40 and 90 degrees when the second brake member engages the disc. The materials of the disc-contacting surfaces of the second brake member and the side surfaces of the disc are chosen for their wear characteristics and to yield an appropriate coefficient of friction. Advantageously, these materials are also selected having a high stiffness so that tangential, radial and axial deformations produced by the braking moment, the engaging force and the contact force, respectively are insignificant in the overall behaviour of the joint. Thus, in the preferred embodiment, the disc is made of stainless steel and the brake member is made of a bronze alloy, neither the disc nor the second brake member having a separate lining material. Typically, the taper angle β of the disc with respect to a plane normal to the knee axis is less than 30° and, preferably, is between 10° and 15°. Selection of the materials of the interengaging surfaces and this taper angle is such that the difference between (1) an angle γ given by γ=tan⁻¹(μ/sin β) and (2) the activation angle, α is between 5° and 45° (μ being the above-mentioned coefficient of friction). This combination, in particular, is preferred as a means of achieving a degree of flexion-moment activation of the stabilising action whilst still allowing the stabilising action to cease as the compressive load on the knee joint is removed whilst a flexion moment is applied. The activation angle should be less than 90°, preferably, and greater than 50°, with 65° to 75° being the most preferred range. The most preferred range of the angular difference between y and a is from 30° to 45°.

Summarising, the amplification of the activation force to produce a substantially increased engagement force in the frictional surfaces is such as to allow the joint to provide sufficient stability in the initial stage of the stance phase, and subsequently allow the stabilising action to decrease as the compressive force on the knee joint is decreased even whilst a flexion moment is applied. This condition determines a relationship between the amplification of the engaging force due to the wedge profile of the disc and the amplification of the activation force due to the activation angle. For the preferred embodiment of this invention, this relationship can be stated as 5°<(α−γ)<45°. The brake formed by the joint is load-sensitive but substantially insensitive to the flexion moment applied to the joint. It is activated and remains activated only when the load applied to the joint is posterior to the activation pivot.

According to a third aspect of the invention, a stabilising prosthetic knee joint for a lower limb prosthesis comprising an upper joint portion for attachment to a thigh part and a lower joint portion for attachment to a shin part, the upper joint portion being rotatable relative to the lower joint portion about a knee axis, wherein the joint further comprises: first and second brake members one of which rotates with the upper joint portion and the other with the lower joint portion, relative rotation between the brake members occurring about an axis of relative rotation, wherein: the first brake member is rotatably mounted on an activation arm which is, itself, pivotally mounted on the joint portion with which the second brake member rotates, such pivoted mounting defining an activation axis substantially parallel to and spaced from the said axis of relative rotation; and the joint is arranged such that application of a compressive force to the joint causes the activation arm to pivot about the activation axis so as to cause the first brake member to bear against the second brake member whereby frictional engagement between the brake members hinders relative rotation therebetween; the joint being further arranged such that when the joint is weight-bearing with the load thereon tending to flex the joint, the forces acting on the first brake member in a plane perpendicular to the knee axis, when in equilibrium, comprise:
(i) a resolved force acting at a distance d from the said axis of relative rotation, being the combination of the load due to a user's weight, a flexion moment resulting therefrom, and any counteracting spring-biasing force, which resolved force acts in a vertical direction when the joint is oriented in its normal user-standing orientation;
(ii) a resultant rotation-resisting force acting at a distance from the axis of relative rotation and having a radial component and a tangential component, the direction in which the force acts being dependent, at least in part, on the coefficient of friction of the interengaging surfaces of the first and second brake members; and
(iii) a reaction force acting at the axis of relative rotation along a line joining the axis of relative rotation and the activation axis; wherein the angle between the rotation-resisting force and the reaction force is sufficient to cause substantial release of the said frictional engagement when the compressive force is removed, even when a flexion moment is applied to the joint.

According to a fourth aspect of the invention, there is provided a weight-activated stabilising prosthetic knee joint comprising upper and lower knee joint components interconnected by a resiliently biased activation member having respective spaced-apart pivotal connections to the said joint components, wherein the knee joint further comprises a brake rotor on one of the components and a brake shoe on the other of the components, the rotor and the shoe having matching inclined surfaces that interengage frictionally in the manner of a wedge in a V-shaped groove in response to a relative approaching movement of the upper and lower components associated with a pivoting movement of the activation member against the resilient biasing when a compressive load is applied to the knee joint, such frictional engagement resisting flexion of the joint, wherein in a medial-lateral view, the geometry of the joint is defined by:

5°<(α−y)<45°

where α is the acute angle between first and second lines in an anterior-posterior plane perpendicular to the knee axis of rotation, the first line being a line passing through the pivot axes of the pivotal connections of the activation member and the second line being a line passing through the pivot axis of the pivotal connection between the activation member and the said knee joint component carrying the rotor and through the centroid of the interengaging surfaces of the rotor and the brake shoe, and where γ=tan ⁻¹(μ/sin β), μ being the coefficient of friction of the interengaging surfaces and β being the inclination of the interengaging surfaces with respect to a plane perpendicular to the knee axis of rotation.

In one embodiment of the invention, both the activation member and the brake shoe are resiliently pre-loaded, the latter providing resilience of the knee joint during the stance phase. The degree of pre-loading of the brake shoe is preferably adjustable, the range of adjustment including a setting in which deflection commences when a compressive load of 70 kgf is applied to the joint in the longitudinal direction. Deflection of the brake shoe from its stop occurs progressively as the compressive load increases beyond that required to overcome the biasing force of the brake member biasing spring. The spring is further compressed and the extent of resilient deflection increases in proportion to the additional force applied, giving an appropriate resilience of the knee joint during the stance phase. Resilience of the joint in the stance phase is, therefore, initially governed by the resilient biasing of the activation member and, when the pre-loading of the brake member spring is overcome, is primarily dependent on the stiffness of that spring. The deflection versus compressive load characteristic, therefore, has two phases of different respective gradients.

In an alternative embodiment, stance phase resilience is provided by allowing limited resiliently biased relative rotation between the brake rotor (the first brake member) and whichever of the upper and lower joint components with which it rotates during flexion of the joint. In this alternative embodiment, the second brake member is fixed with respect to the other of the joint components, at least during operating of the joint.

According to a fifth aspect of the invention, there is provided a stabilising knee joint having a knee brake capable of locking when a compressive load is applied to the joint, and comprising a stance cushioning device arranged such that substantially no resilient flexion is produced by the cushioning device until the applied compressive load reaches at least 25% of the compressive load required to produce 3° of knee flexion.

According to a sixth aspect of the invention, there is provided a weight-activated stabilising prosthetic knee joint comprising an upper component for attachment to a thigh part and a lower component for attachment to a shin part, the lower component having rotational motion with respect to the upper component with either a monocentric or a polycentric movement, wherein the joint further comprises a friction brake in which the resistance to prevent knee flexion during the stance phase of gait is proportional to a compressive force applied to the joint, and such that subsequent decrease of the compressive force to the joint causes decrease of the said frictional engagement even when a flexion moment is applied, wherein the joint further provides resilience of the knee joint during the stance phase of gait.

The invention will now be described by way of example with reference to the drawings in which:

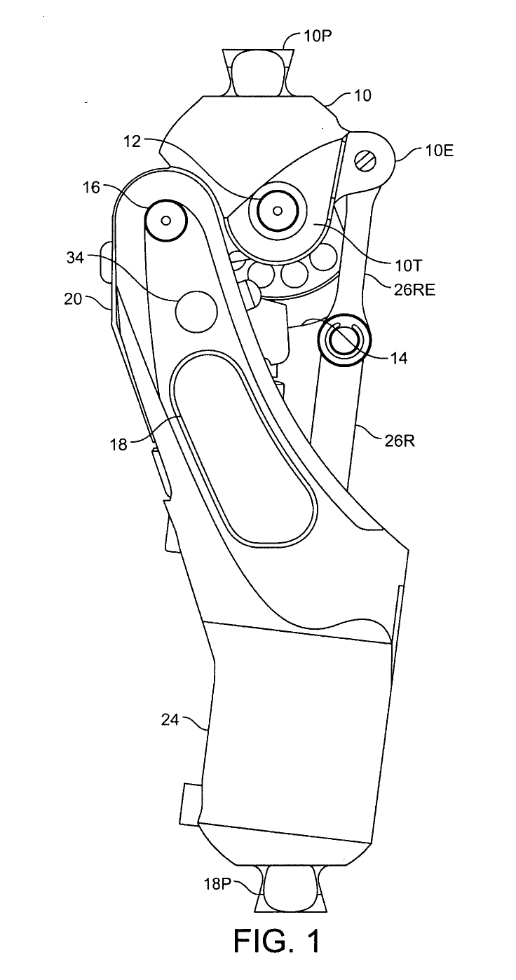

FIG. 1 is a diagrammatic side view of a first knee joint in accordance with the invention;

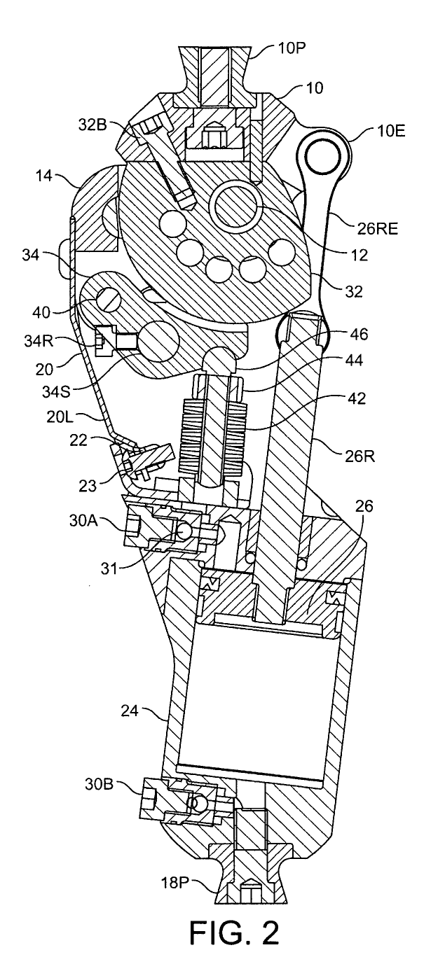

FIG. 2 is a cross-section through the first knee joint on a sagittal plane passing through the middle of the joint;

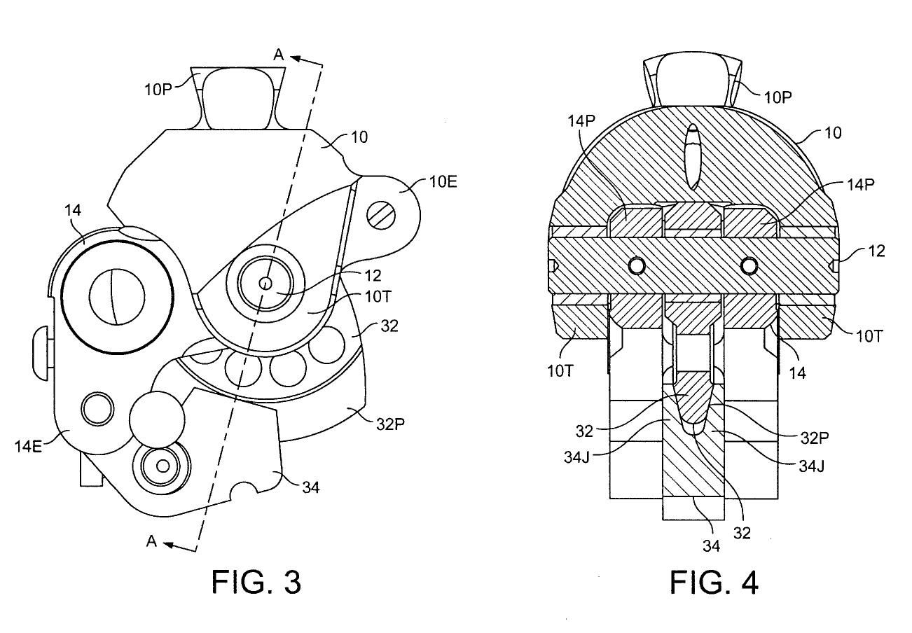

FIG. 3 is a side view of parts of the joint;

FIG. 4 is a cross-section on the line A-A appearing in FIG. 3;

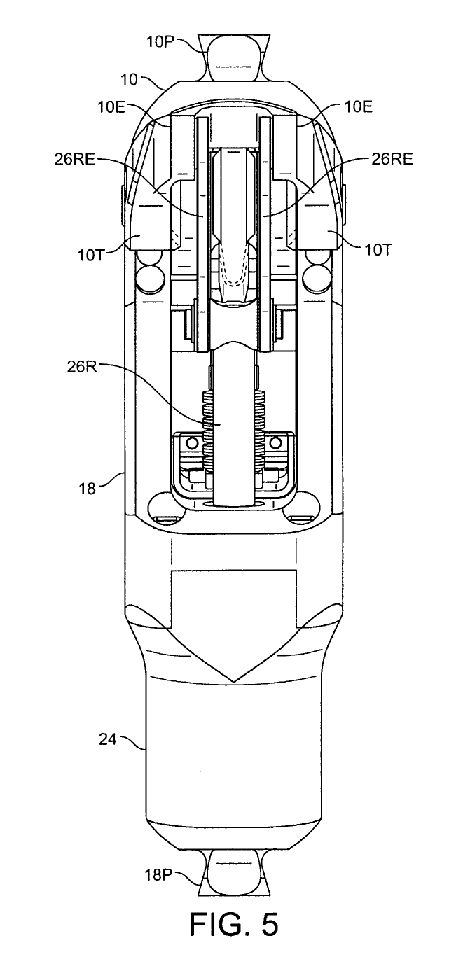

FIG. 5 is a posterior view of the first knee joint;

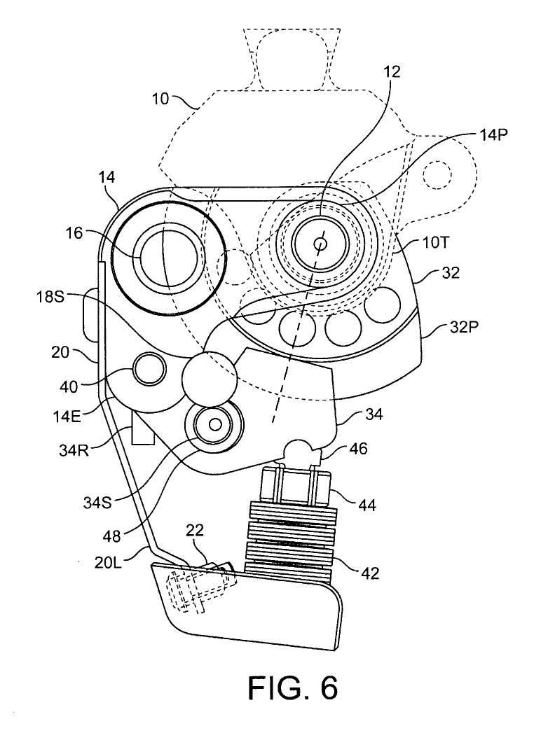

FIG. 6 is a side view of parts of the first knee joint associated with a lower component thereof, with an upper component thereof shown in phantom;

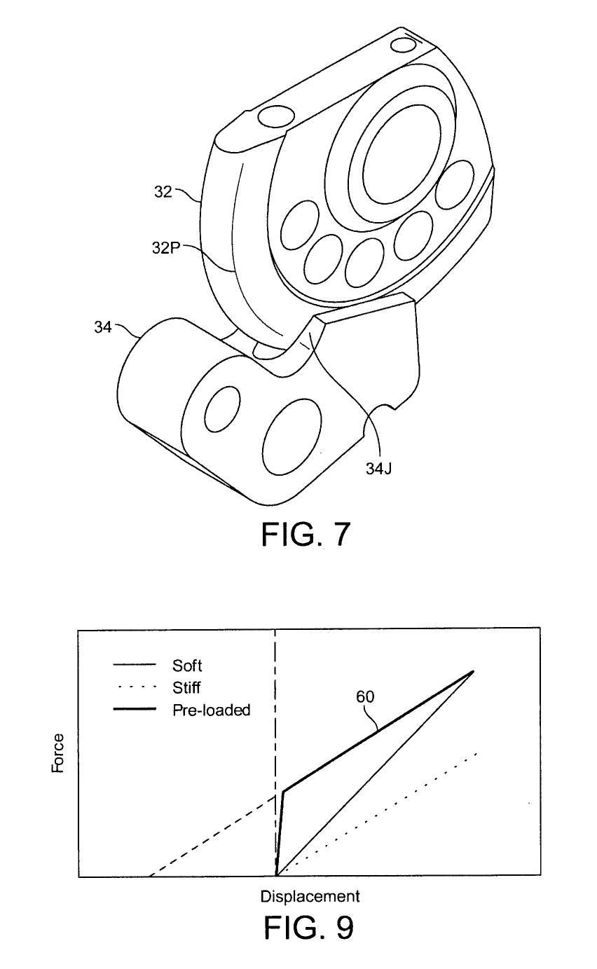

FIG. 7 is a perspective view of a brake forming part of the knee joint of FIG. 1;

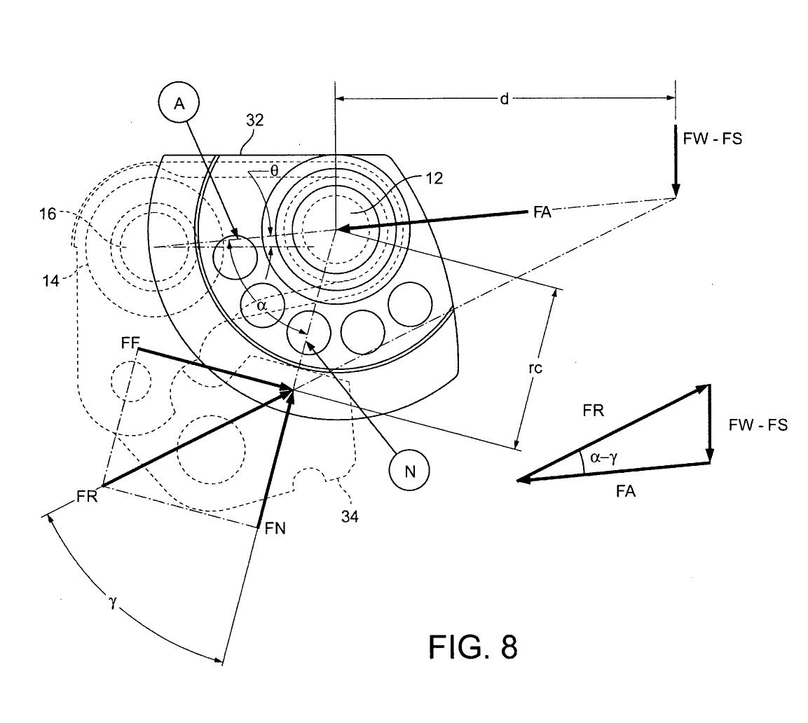

FIG. 8 is a forces diagram showing forces acting on part of the brake during knee stabilisation;

FIG. 9 is a graph showing a knee flexion characteristic when the knee joint is locked;

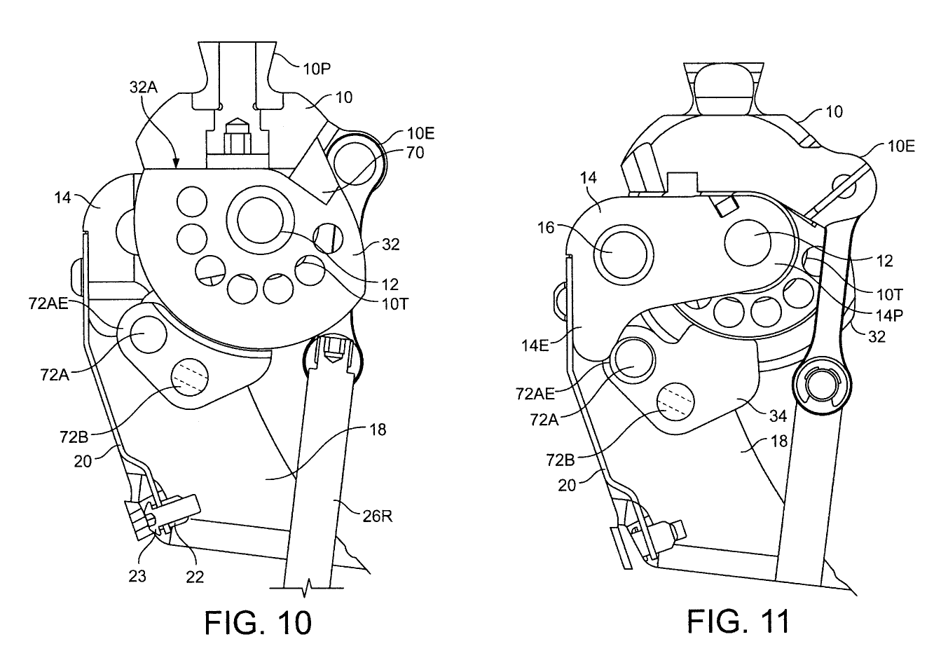

FIG. 10 is a cross-section through a second knee joint in accordance with the invention, the cross-section being on a sagittal plane passing through the middle of the joint; and

FIG. 11 is a second cross-section through the second knee joint, this section being on a plane parallel to but offset from the sagittal plane of the cross-section of FIG. 10.

Referring to FIGS. 1 and 2, a stabilising knee joint in accordance with the invention comprises an upper knee component in the form of a knee chassis 10 having a proximally directed pyramid connector 10P for interfacing with an alignment coupling and stump socket (not shown). As shown in FIGS. 3 to 6, the knee chassis 10 is constructed as a trunnion, the side members 10T of which house a medial-lateral shaft 12 that is supported in the posterior end portions 14P of an activation arm or lever 14. The knee chassis 10 is free to pivot with respect to the activation arm 14 about the axis of the shaft 12 which carries a bearing, the shaft axis thereby forming the knee axis of rotation. Structural loads on the knee joint, therefore, are borne by the shaft 12.

As shown in FIG. 1, the activation arm 14 is pivotally mounted by means of a second shaft 16 in a lower knee component formed as a shin carrier 18 having a distally directed pyramid connection interface 18P for connection to shin and foot components (not shown). In order to bias the posterior end portions 14P of the activation arm 14 upwardly, a downwardly depending leaf spring 20 is secured to an anterior nose portion of the activation arm 14, the lower end portion 20L of the leaf spring engaging a stop 22 attached to the carrier 18. Accordingly, in the absence of a compressive load on the knee joint applied between the connection interfaces 10P and 18P, the activation arm is biased against and abuts activation arm stop members 18S on the carrier 18, as seen most clearly in FIGS. 2 and 6, the ears 14E of the activation arm abutting the stop members 18S. The amount of pre-load in the leaf spring 20 can be modified by means of the adjustment screw 23 in contact with the stop 22, and which has a threaded connection with the lower end portion 20L of the leaf spring 20 as seen on FIGS. 2 and 6.

Referring now to FIGS. 2 and 5, the carrier 18 incorporates a piston and cylinder assembly comprising a cylinder 24 that houses a reciprocable piston 26 having a piston rod 26R extending posteriorly with respect to the knee axis. A piston rod extension comprising a parallel pair of extension struts 26RE pivotally attached to the proximal end of the piston rod 26R and spaced apart in the medial-lateral direction are pivotally attached to posterior ears 10E of the knee chassis 10. This piston and cylinder assembly is a pneumatic device in this embodiment, having individually adjustable valves 30A, 30B in passages formed in the carrier 18 and the cylinder 24 and in communication with cylinder spaces respectively above and below the piston 26. Each valve is an arrangement of an adjustable restriction and a non-return feature 31, allowing the controlled flow of air into and out of each pneumatic chamber defined by the piston. Thus, relative rotation of the knee chassis 10 and the shin carrier 30 is pneumatically controlled, the degree of damping and pneumatic resilience being individually adjustable for flexion and extension rotation.

Knee stabilisation is performed by a knee brake, the main components of which are shown in isolation in FIG. 7, and in relation to the above-described elements in FIGS. 2 and 6. Referring to FIGS. 2, 4, 5 and 6 together, the brake comprises a first brake member in the form of a part-circular brake disc 32 that rotates with the knee chassis 10 as the latter rotates with respect to the carrier 18, and a second brake member or brake shoe 34 movably attached to the activation arm 14 and associated with the carrier 18. Since the brake disc 32 is mounted to the knee chassis or upper knee component 10 which, in turn, is mounted on the end portion 14P of the activation arm 14, and since the activation arm 14 is rotationally biased by the leaf spring 20, the brake disc 32 is resiliently biased away from the second brake member or brake shoe 34. Application of a compressive load to the knee joint causes the activation arm 14 to pivot on the carrier 18 so that the brake disc 32 approaches and engages the brake shoe 34, friction between the disc 32 and shoe 34 hindering relative rotation therebetween and, hence, flexion of the knee joint. In this embodiment, the brake disc 32 is pivotally supported with respect to the carrier 18 by virtue of the pivotal mounting of the activation arm 14.

In this embodiment of the invention, the disc 32 is rigidly attached to the upper component or knee chassis 10 by its rigid mounting on the knee axis shaft 12 so that the disc centre coincides with the knee axis of rotation. In radial cross-section, the disc is outwardly tapered, as best seen in FIGS. 4 and 7, the taper being of constant cross section over the whole range of angles that the periphery of the disc 32 subtends at the disc centre. The brake shoe 34 is pivotally secured in the activation arm 14 beneath the disc 32 and has medial and lateral jaws 34J with inclined inner surfaces, the space between the jaws 34J receiving the tapered peripheral portion 32P of the disc 32. It should be noted that the inclination of the inner surfaces of the jaws 34J in a radial cross-section containing the disc axis of rotation corresponds to the inclination of the tapered or peripheral edge portion 32P of the disc. Furthermore, these inner surfaces correspond to the edge portion 32P in both radius of the curvature and centre of curvature. It will be noted that structural loads on the knee joint are carried by the pivotal interconnections of the chassis 10, activation arm 14 and the carrier 18, rather than the brake components.

The sides of the groove formed between the jaws 34J of the brake shoe 34 form disc-contacting surfaces, the brake shoe 34 being located so that it receives the tapered peripheral portion 32P of the disc 32 and so that when the activation arm 14 is deflected from its position against the stop 22 by application of a compressive load to the knee joint, the brake shoe disc-contacting surfaces engage the correspondingly inclined side surfaces of the disc peripheral portion 32P, as best seen in FIG. 4. The circumferential extent of the disc contacting surfaces is such that they subtend an angle of between 25° and 45° at the axis of the disc (the knee axis). Pivotal mounting of the brake shoe 34 on the activation arm 14 is effected by means of a pin 40 housed in the activation arm 14.

It is possible to define a centre of action of the brake shoe 34 on the disc 32 which is the centroid of the disc contacting surfaces where they overlap the tapered peripheral surface portion 32P of the disc 32. The pivot axis defined by the brake shoe pivot pin 40 is offset with respect to a line joining the disc axis and this centroid, in this case anteriorly with respect to the line such that, once the disc 32 contacts the brake shoe 34, continuing pivoting of the activation arm 14 about its pivot shaft 16 in the carrier 18 causes the brake shoe to pivot downwardly about the axis defined by the pivot shaft 40 against a compression spring 42 acting against an outer face of the brake shoe 34 to bias it towards the disc 32. The compression spring 42 preloads the brake shoe 34 against the stop member located on the opposite side of the brake shoe 34 from the spring 42, in this case the same stop member 22 limiting movement of the activation arm 14. The amount of pre-load exerted by the compression spring 42 can be modified by means of an adjusting nut 44 which has a threaded connection with a spring guide 46.

In a variant of the illustrated and described embodiment of the invention, the brake shoe 34 is pivotally or rigidly attached to the shin carrier 18. However, a resiliently displaceable brake shoe, as described, provides resilience of the knee joint during the stance phase of the walking cycle.

By resiliently preloading the brake shoe 34 against the stop 22, a small clearance is maintained between the brake shoe 34 and the disc 32 when the joint is unloaded so that the disc 32 and knee chassis 10 are free to rotate about the knee axis defined by the shaft 12. This clearance is adjustable by means of a shaft 34S that is free to rotate inside the brake shoe 34 when a set screw 34R is released. The shaft 34S has eccentric bosses that protrude at either side of the brake shoe 34, on which rollers 48 are free to rotate. By changing the angular position of the shaft 34S relative to the brake shoe 34, the position of the rollers 48 relative to the brake shoe 34 is modified, allowing more or less clearance between the disc 32 and the brake shoe 34. When a compressive load is applied to the joint, a moment is produced about the activation pivot defined by the second shaft 16 which, when it is high enough to overcome the preload moment created by the activation arm biasing spring 20, causes the activation arm 14 to rotate and the disc 32 to engage the disc-contacting surfaces of the brake shoe 34.

The contact force between the brake disc and the brake shoe is amplified several times by the acute angle of the wedge geometry of the disc peripheral portion 32P and the correspondingly inclined surfaces of the brake shoe jaws 34J, as well as by the positioning of the activation pivot defined by shaft 16, as will be explained in more detail below. Contact friction between the disc-contacting surfaces of the brake shoe 34 and the peripheral portion 32P of the disc 32 resists rotation of the disc relative to the shoe thereby to provide stance control substantially proportional to the compressive load applied to the joint by the patient.

At the late stage of the stance phase of gait, as the patient's body weight shifts forwardly and moves towards the other limb, at which time the load applied to the knee joint decreases or moves forwardly relative to the activation pivot, the activation moment progressively decreases until it is less than the activation arm preload moment generated by the leaf spring 20. At the same time, the contact forces between the disc and the shoe are reduced, reducing the frictional force applied to the disc by the shoe until a point is reached at which the disc 32 is no longer in contact with the shoe 34, the disc being released smoothly and progressively to a state in which the knee is free to rotate again, allowing completion of the stance phase rollover and initiation of knee flexion to start the swing phase of the walking cycle.

The joint mechanism produces a mechanical advantage in causing the brake members to bear against each other by the application of two different and independent principles. In the first place, the corresponding surfaces of the brake members (i.e. the disc 32 and the shoe 34) are inclined so as they form an acute angle with the direction in which they move relative to each other, in the same configuration as a wedge. Consequently, the contact force between the interengaging surfaces is greater than the engaging force that brings them into contact. In the second place, the direction the engaging movement is not parallel to the direction of the activation movement, but forms an acute angle with it. As a consequence, the engaging force is greater than the activation force. Both means of amplification combine together to produce an overall mechanical advantage that generates a contact force several times higher than the activation force.

The selection of materials for the interengaging surfaces of the disc 32 and brake shoe 34 is governed by the requirements of durability and a suitable coefficient of friction, as well as consistency of application and release of the frictional knee-stabilising force. The applicants have found that the most acceptable properties are obtained if both surfaces are made of rigid material, the disc-contacting surfaces preferably being softer than the material of the disc. In the preferred embodiment, the disc is made of stainless steel and the brake shoe is made of a non-ferrous bearing material such as aluminium or, more preferably, a alloy with no lining. However, in other embodiments a lining may be provided for the jaws 34J of the brake shoe 34 to provide the required bearing material. The above combination produces sufficient friction yet avoids sticking of the disc-contacting surfaces on the disc 32. The coefficient of friction using stainless steel and a bronze alloy is in the region of 0.25. A suitable angle of inclination of the interengaging surfaces with respect to a plane normal to the disc rotation axis is 12°. Smaller angles can be used if harder materials are used for the disc and the shoe. Larger angles of inclination can be used but, in practice, an angle greater than 30° or 35° with respect to the central plane of the disc yields insufficient amplification of the compressive loading of the disc in the direction of the brake shoe.

The applicants have found that it is possible to achieve a progressive release of the braking action at the end of the walking cycle stance phase without locking of the joint and without residual locking brought about by a knee flexion moment.

“Sticking” of the joint, and a sharp transition between a free state and a fully locked state of the knee joint causes discomfort for the amputee and unacceptable noise, as well as requiring compensatory muscular actions by the amputee to release the limb, thereby altering the gait pattern and shifting the body weight forwardly too quickly and onto the other limb. A progressive release of the stabilising action, coupled with the ability to stabilise the knee not only on level ground, but also on inclined surfaces and at different walking speeds, is achievable using the described and illustrated construction. Parameters which influence progressive release include the activation angle α, the wedge angle β of the disc 32, the different surface hardnesses of the disc 32 and the brake shoe 34, and the coefficient of friction.

Referring to FIG. 8, the activation angle α, is the angle between a first line N passing through the knee axis and the above-mentioned centroid of the interengaging surfaces and a second line A passing through the axis of rotation of the disc on the activation arm 14 (defined by shaft 12) and the pivot axis of the activation arm (defined by the second shaft 16).

For further explanation, reference is now made to the force diagram of FIG. 8.

When considered as a free body, the disc 32 experiences forces and moments. In the diagram of FIG. 8, FW is the force due to the patient's weight that, for the sake of this analysis, is considered vertical. FS is the force produced at the disc by the preload of the leaf spring 20 (FIG. 1), opposing in part the action of FW. Again, for this analysis, FS will be considered vertical, so that the resultant of FW and FS is also vertical. FA is the force directed along the activation arm 14 (line A in FIG. 1), which is at an angle θ to the horizontal. FN is the normal force between the disc 32 and the shoe 34 in the radial direction (line N). This force is amplified by the wedge effect of the disc geometry, so that the contact force is (1/sin β) times FN. FF is the friction force corresponding to the contact force and the coefficient of friction μ. The resultant of FN and FF is FR, directed at an angle of γ=tan ⁻¹(μ/sin β) with respect to FN. The flexion moment on the knee joint can be replaced by shifting the vertical force backwards to a distance d from the knee axis, as shown in FIG. 8.

For the disc to be in equilibrium, the three forces (FW-FS), FA and FR must be concurrent so that they sum to zero. This defines the distance d and hence the maximum flexion moment that the disc brake is able to counteract. Based on the assumptions of this analysis, the value of d is computed as

$$d = \frac{{r}_{c} \cdot sin γ \cdot cos θ}{sin ( α - γ )}$$

From this expression and the free body diagram, it can be stated that the distance d is:
Proportional to the contact radius r_{c}, of the disc 32;
Increases with increasing angle γ, and, therefore, increases with increasing coefficient of friction p and with decreasing wedge angle β; and
Increases as the angle θ of the activation arm with respect to the horizontal decreases.

A significant parameter is the difference between the activation angle γ and the angle γ of the resultant frictional force vector FR with respect to the above-mentioned first line joining the knee axis with the centroid of the interengaging surfaces. As α approaches γ, the difference reduces and the term sin (α-γ) approaches zero. If (α=γ), the brake is self-locking and can, in theory, withstand the force at any distance from the knee axis. This is undesirable, as the knee is then unable to initiate flexion in the early stages of the swing phase.

With a knee joint in accordance with the present invention, it is possible to achieve sufficient stability for normal everyday use, yet the joint releases readily in the early part of the swing phase when some of the body weight is still applied to the joint. Such “clean release” is to be distinguished from the self-locking properties of some stabilised knee joints which require the whole of the patient's body weight to be transferred to the contralateral limb for flexion to take place.

The applicants have found that a knee joint constructed in accordance with the invention can be capable of withstanding a moment of 50 Nm with a compressive load of 1000 N applied.

Preferred values of the above-mentioned parameters and preferred ranges are as follows:

r_{c}: 24 mm; 15 mm to 35 mm.

α: 70°; 55° to 90°.

β12°; 5° to 35°.

μ: 0.25; 0.1 to 0.40.

θ: 5°; 0 to 15°

These limits are mutually exclusive; i.e. this specification includes within its scope ranges of values which include only one of the two limits in each case. At the other ends of the ranges, different values may be appropriate.

As described above, resilient mounting of the brake shoe 34 provides a degree of resilience in the joint during the stance phase, such resilience being apparent when the joint is locked by a small amount of resilient knee flexion. The knee flexion characteristic is illustrated in FIG. 9, which is a graph indicating the compressive load applied to the joint for different degrees of knee flexion. During the early part of the stance phase (peak load occurring at heel strike), the disc 32 makes contact with the brake shoe 34 and both pivot downwardly about the shaft 16 against the compression spring 42. The resulting vertical compliance of the joint reduces the impact force produced at heel strike and also reduces the effective leg length, making the gait more natural and energy efficient as a result of the smaller vertical displacement of the patient's centre of gravity. The location of the pivot axis 34, close to the periphery of the disc 32 and offset anteriorly with respect to the first line A projected beyond the centroid of the interengaging surfaces, optimises the flexion behaviour during the stance phase without substantially increasing the braking force exerted by the joint.

Referring to FIG. 9, the preloading of the brake shoe spring 42 produces a stepped characteristic 60 in the force versus displacement graph. In the initial part of the curve, i.e. when the applied force is insufficient to overcome the preload force of the spring, the behaviour is that of an infinitely stiff spring. However, as soon as the pre-load level of the spring is achieved, the brake shoe 34 loses contact with the stop 22 and begins to behave like a free spring which has a comparatively soft spring rate, despite the load having, by then, reached relatively high values. In the preferred embodiment of the present invention, the spring preload by means of the adjusting nut 44 (FIG. 6) is set at about 70% to 80% of the patient's body weight so that, when the patient is standing still on both feet, no resilient flexion is evident, the joint being substantially infinitely stiff. However, when more of the body weight is transferred to the prosthetic limb, the joint behaves like a comparatively soft spring, allowing a flexion range of up to 5° when, for instance, 120% body weight is applied.

A similar principle is used for the leaf spring 20, the initial deformation of the leaf spring being set accordingly by means of the adjustment screw 23.

In alternative variants, servo pneumatic valves can be used in the piston and cylinder assembly to provide consistent damping for a different cadence of the amputee. A servo valve typically uses a floating needle valve member that is under the pressure of light spring actuated by the air pressure inside the chamber and restricted by the level of adjustment and travel set to match a particular amputee's walking pattern.

The valve arrangement can include a servo stepper motor as described in GB-A-2280609. Microprocessor control may be used to provide variable swing phase characteristics for different cadences as described in GB-A-2334891 and WO-A-2007/110585.

Adjustment of the leaf spring 20 (FIG. 2) can be performed by the user to match the compression load required by the user for stability in differing environments or according to their ability to control the stance phase of walking cycle. The adjusting means may restrict the travel and hence the preload caused by the stiffness of the spring. The use of a composite leaf spring is preferred for optimum control of the rate of release of the pre-compressed force, and hence the resulting reaction in controlling the rate of pre-load which enhances the progressive release of this stabilising mechanism. In this way, improved proprioception of the knee action can be provided for the user, enabling better control and closer matching of the action of the prosthesis to the user's need.

Referring to FIGS. 10 and 11, in a further, preferred knee joint in accordance with the invention, the second brake member or brake shoe 34 is fixed to the shin carrier 18 rather than being pivotally and resiliently mounted. As in the first knee joint described above with reference to FIGS. 1 to 6, the knee chassis 10 is supported on the shaft 12 by medial and lateral trunnion side members 10T, one of which is visible in FIGS. 10 and 11. In this case, stance phase resilience is provided by allowing limited resiliently biased relative rotation between the brake disc 32 and the knee chassis 10. Specifically, both the knee chassis 10 and the brake disc 32 are free to rotate independently of each other on the medial-lateral shaft 12 defining the knee axis. The shaft is fixed in the posterior end portion 14P of the activation arm 14 (see FIG. 11). Relative rotation between the brake disc 32 and the knee chassis 10 is limited, on the one hand, by the abutment of part of the chassis 10 which is spaced from the knee axis defined by the shaft 12 against an abutment surface 32A of the brake disc 32, as shown in FIG. 10. This limits relative rotation of the chassis 10 and disc 32 in the direction of knee joint extension. Relative rotation of the chassis 10 and the disc 32 in the direction of knee joint flexion is limited by a resilient element 70, here in the form of an elastomeric buffer interposed between the chassis 10 and the brake disc 32 at a location spaced from the knee axis, as shown in FIG. 10. The buffer 70 preloads the knee chassis 10 to cause it to bear against the abutment surface 32A of the brake disc 32 when there is no compressive load or flexion moment on the joint. However, when the load is sufficient to produce a flexion moment which overcomes the resilient biasing force of the buffer 70, the knee chassis 10 rotates with respect to the brake disc 32 to produce a small amount of knee flexion, the degree of flexion increasing with increasing flexion moment. In this embodiment, the abutment surface 32A of the brake disc 32 and the knee chassis part 10A which engages it are located anteriorly with respect of the knee axis, whereas the resilient biasing of the chassis 10 with respect to the disc 32 in the direction of knee flexion is performed by the buffer 70 located posteriorly with respect to the axis 12.

The brake shoe 34 is fixed to the shin carrier 18 by two medial-lateral pins 72A, 72B.

One of the pins 72A has at least one eccentric portion 72AE which acts as a stop member for the activation arm 14. The stop position of the activation arm 14 with respect to the carrier 18 is adjustable by rotating the pin 72A in the carrier 18. In this way, the initial, no-load gap between the brake disc 32 and the brake shoe or receiver 34 can be preset.

1. A stabilising prosthetic knee joint for a lower limb prosthesis, comprising an upper component for attachment to a thigh part and a lower component for attachment to a shin part, the upper component being pivotable relative to the lower component, wherein the joint further comprises a first brake member that moves with one of the components and a second brake member associated with the other component, the first and second brake members having opposing surfaces, and wherein the first brake member is resiliently biased away from the second brake member such that when a compressive force is applied against the resilient bias to the joint causing enough moment to overcome the bias, the first brake member bears against the second brake member whereby frictional inter-engagement of the opposing surfaces resists flexion of the joint, and such that subsequent decrease of the moment caused by the compressive force below the level of the resilient bias, by reduction in the force magnitude or by change in the position of the force relative to the joint, causes substantial release of the said frictional engagement even when a flexion moment is applied to the joint.

2. A stabilising prosthetic knee joint for a lower limb prosthesis, comprising an upper component for attachment to a thigh part and a lower component for attachment to a shin part, the upper component being pivotable relative to the lower component, wherein the joint further comprises a first brake member that moves with one of the components and a second brake member associated with the other component, the first and second brake members having opposing surfaces, and wherein the first brake member is mounted so as to be movable towards the second brake member against resilient biasing means in response to the application of a compressive force to the joint to cause the first brake member to bear against the second brake member whereby frictional inter-engagement of the opposing surfaces resists flexion of the joint, and wherein the mechanism of the joint is so configured that a resultant force imposed on the first brake member by the second brake member when the said compressive force is applied to the joint acts in a direction that allows substantial release of the said frictional engagement when the compressive force is removed, even when a flexion moment is applied to the joint.

3. A knee joint according to claim 1, wherein the mechanism of the joint is configured so as to produce a mechanical advantage in converting a brake activation force into a brake member engagement force causing the first brake member to bear against the second brake member, such mechanical advantage being produced substantially independently of any flexion moment applied to the joint.

4. A knee joint according to claim 1, wherein the brake members are configured and supported so as to produce a mechanical advantage in generating an engagement force causing the first brake member to bear against the second brake member, the mechanical advantage being such that the activation force is the dominant component, at all times during use of the joint, of the total force causing the brake members to bear against each other.

5. A knee joint according to claim 3, wherein the mechanical advantage is produced by the orientation of the said opposing surfaces.

6. A knee joint according to claim 1, wherein one of the brake members is a brake disc that is of tapered cross-section and the other brake member is a channel member having a channel defined between jaws with disc-contacting surfaces substantially parallel to respective side surfaces of the disc.

7. A knee joint according to claim 6, wherein the brake disc rotates with the upper component of the joint and is centred on a knee axis defined by an axis of rotation of the upper component relative to the lower component.

8. A knee joint according to claim 1, wherein the first brake member is mounted on a resiliently deflectable activation arm, the orientation and mounting of the arm and the brake members being such that when a compressive load is applied to the joint, the arm is deflected so as to bring the opposing surfaces of the brake members into frictional engagement in order to resist flexion of the joint.

9. A knee joint according to claim 8, wherein the activation arm is pivotally mounted on the said one of the upper and lower components and has an outer end portion that carries the first brake member and the other of the upper and lower components, the pivotal mounting of the activation arm defining an activation axis located anteriorly with respect to a knee axis defined by an axis of rotation of the upper component relative to the lower component, whereby the opposing surfaces of the brake members frictionally engage only when the compressive load acts along a line to the posterior of the activation pivot and wherein the included angle (hereinafter the “activation angle”) between (a) a first line passing through the axis of the relative rotation of the first brake member and activation arm and the centroid of the interengaging surfaces of the brake members and (b) a second line passing through the axis of rotation of the first brake member on the activation arm and the pivot axis of the activation arm is between 40 degrees and 90 degrees when the second brake member engages the disc.

10. A knee joint according to claim 1, wherein the second brake member is resiliently mounted in one of the upper and lower components.

11. A knee joint according to claim 10, wherein the second brake member is a lever pivotally mounted to one of the upper and lower components, the associated brake member pivot axis being substantially parallel to the knee axis and spaced from the first line.

12. A knee joint according to claim 9, wherein the materials of the disc-contacting surfaces of the second brake member and the side surfaces of the disc, and the taper angle β of the disc with respect to a plane normal to the knee axis are such that the difference between (i) an angle γ given by γ=tan ⁻¹(μ/sin β), where μ is the coefficient of friction yielded by the said materials, and (ii) the activation angle, α, is between 5° and 45°.

13. A stabilising prosthetic knee joint for a lower limb prosthesis comprising an upper joint portion for attachment to a thigh part and a lower joint portion for attachment to a shin part, the upper joint portion being rotatable relative to the lower joint portion about a knee axis, wherein the joint further comprises:
first and second brake members one of which rotates with the upper joint portion and the other with the lower joint portion, relative rotation between the brake members occurring about an axis of relative rotation, wherein:
the first brake member is rotatably mounted on an activation arm which is, itself, pivotally mounted on the joint portion with which the second brake member rotates, such pivoted mounting defining an activation axis substantially parallel to and spaced from the said axis of relative rotation; and
the joint is arranged such that application of a compressive force to the joint causes the activation arm to pivot about the activation axis so as to cause the first brake member to bear against the second brake member whereby frictional engagement between the brake members hinders relative rotation therebetween;
the joint being further arranged such that when the joint is weight-bearing with the load thereon tending to flex the joint, the forces acting on the first brake member in a plane perpendicular to the knee axis, when in equilibrium, comprise:
(i) a resolved force acting at a distance d from the said axis of relative rotation, being the combination of the load due to a user's weight, a flexion moment resulting therefrom, and any counteracting spring-biasing force, which resolved force acts in a vertical direction when the joint is oriented in its normal user-standing orientation;
(ii) a resultant rotation-resisting force acting at a distance from the axis of relative rotation and having a radial component and a tangential component, the direction in which the force acts being dependent, at least in part, on the coefficient of friction of the interengaging surfaces of the first and second brake members; and
(iii) a reaction force acting at the axis of relative rotation along a line joining the axis of relative rotation and the activation axis;
wherein the angle between the rotation-resisting force and the reaction force is sufficient to cause substantial release of the said frictional engagement when the compressive force is removed, even when a flexion moment is applied to the joint.

14. A knee joint according to claim 13, wherein the said angle is greater than 5°.

15. A knee joint according to claim 14, wherein the said angle is less than 45°.

16. A weight-activated stabilising prosthetic knee joint comprising upper and lower knee joint components interconnected by a resiliently biased activation member having respective spaced-apart pivotal connections to the said joint components, wherein the knee joint further comprises a brake rotor on one of the components and a brake shoe on the other of the components, the rotor and the shoe having matching inclined surfaces that interengage frictionally in the manner of a wedge in a V-shaped groove in response to a relative approaching movement of the upper and lower components associated with a pivoting movement of the activation member against the resilient biasing when a compressive load is applied to the knee joint, such frictional engagement resisting flexion of the joint, wherein in a medial-lateral view, the geometry of the joint is defined by:
5°<(α-γ)<45°
where α is the acute angle between first and second lines in an anterior-posterior plane perpendicular to the knee axis of rotation, the first line being a line passing through the pivot axes of the pivotal connections of the activation member and the second line being a line passing through the pivot axis of the pivotal connection between the activation member and the said knee joint component carrying the rotor and through the centroid of the interengaging surfaces of the rotor and the brake shoe, and where γ=tan ⁻¹(μ/sin β), μ being the coefficient of friction of the interengaging surfaces and β being the inclination of the interengaging surfaces with respect to a plane perpendicular to the knee axis of rotation.

17. A stabilising knee joint having a knee brake capable of locking when a compressive load is applied to the joint, and comprising a stance cushioning device arranged such that substantially no resilient flexion is produced by the cushioning device until the applied compressive load reaches at least 25% of the compressive load required to produce 3° of knee flexion.

18. A knee joint according to claim 17, wherein the stance cushioning device is arranged such that substantially no resilient flexion is produced by the cushioning device until the applied compressive load reaches at least 50% of the compressive load required to produce 3° of knee flexion.

19. A weight-activated stabilising prosthetic knee joint comprising an upper component for attachment to a thigh part and a lower component for attachment to a shin part, the lower component having rotational motion with respect to the upper component with either a monocentric or a polycentric movement, wherein the joint further comprises a friction brake in which the resistance to prevent knee flexion during the stance phase of gait is proportional to a compressive force applied to the joint, and such that subsequent reduction of the compressive force applied to the joint causes substantial release of the said frictional engagement even when a flexion moment is applied, wherein the joint further provides resilience of the knee joint during the stance phase of gait.
